(19) United States
(12) Patent Application Publication
(10) Pub. No.: US 2008/0312575 A1
(43) Pub. Date: Dec. 18, 2008

(54) Haemofilters for Blood Detoxification

(76) Inventors: Giorgio Graziani, Milano (IT); Annamaria Naggi, Legnano (Milano) (IT); Giangiacomo Torri, Milano (IT)

Correspondence Address: DICKSTEIN SHAPIRO LLP, 1825 EYE STREET NW, Washington, DC, 20006-5403, US

(21) Appl. No.: 11/996,048

(22) PCT Filed: Jul. 27, 2006
(86) PCT No.: PCT/IT2006/000583

§ 371 Date: Jun. 18, 2008

(30) Foreign Application Priority Data
Jul. 28, 2005 (EP) 054255561.7

(51) Int. Cl. A61M 1/34; B01J 20/22; B01J 20/26

(52) U.S. Cl. 604/5.02; 502/404; 502/402; 604/5.04

(57) ABSTRACT

The present invention regards the use of hemofilters for the removal of bacterial toxins (lipopolysaccharides) from the blood, said hemofilters comprising a solid support to which cyclodextrins are covalently bonded. The solid support can be a fabric or non-woven fabric or a polymeric resin obtained by means of cross-linking of the cyclodextrins with appropriate cross-linking agents, for example epichlorohydrin.

The present invention regards the use of supported cylcodextrins for the detoxification of the blood.

The sepsis syndrome is a serious complication of the infection by gram-negative germs.

Frequently, this syndrome involves all organs and apparatuses, leading to multi-organ insufficiencies which require the applications of artificial aids (automatic respirator, artificial kidney, heart stimulators). One particularly feared and widespread complication of sepsis is septic shock, which can lead to death.

Sepsis is triggered by the liberation of the exotoxins and endotoxins coming from the outer capsule of bacteria. Such substances are lipopolysaccharides which, upon entering into circulation, trigger a series of reactions involving the immune system in its humoral and cellular components. The situations most at-risk for this disease include: serious traumas perforations of intestines, upper abdominal surgery operations, especially in diabetic or immunodepressed elderly patients.

The reaction of the organism to the endotoxins frequently causes a series of responses involving the microcirculation of the organs (septic cascade).

Such reaction, while considered a defense of the organism at the bacterial invasion, is often abnormal, inducing the production of numerous inflammation mediators (cytokines and other microproteins), responsible for the self-amplification process of the response, which causes serious damage to organs and apparatuses.

Antibiotic therapy and the substitution of the damaged organs with artificial aids do not always succeed in stemming the septic cascade.

The most effective treatment of sepsis is therefore the prevention of the invasion of the organism by the bacterial toxins, before these induce the septic cascade.

The lipopolysaccharides (LPS) are characteristic components of the cellular membrane of the gram-negative bacteria; they are not present in gram-positive bacteria.

These are molecules composed of a hydrophobic lipid chain, which is responsible for the toxic properties, a hydrophilic central polysaccharide chain and a hydrophilic O-polysaccharide side chain, which is specific for each bacterium strain.

The lipopolysaccharides tend to form aggregates of various size, in particular micelles of about 1,000 KDa weight, in aqueous solvent. Consequently, these are not separable from the blood by means of ultrafiltration, since they do not pass through the pores of the membrane.

Over the years, numerous blood detoxification techniques have been developed, which however have the disadvantage of not being very effective and requiring very high costs particularly due to the employed materials. For example, hemofilters are available comprising antibiotics capable of detoxifying the blood. Nevertheless, such filters have an enormous cost.

The aforesaid problems are resolved by the use of a hemofilter as outlined in the attached claims.

The invention will be explained in detail below with reference to the following figures:

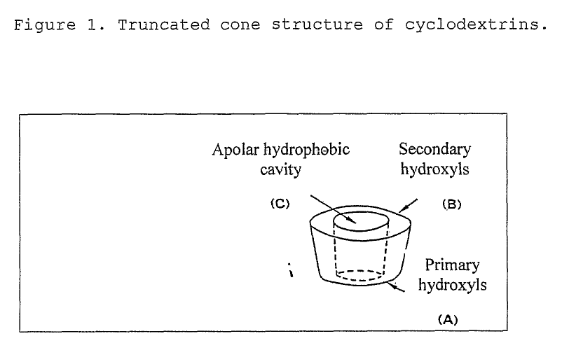

FIG. 1 shows the truncated cone structure of cyclodextrins;

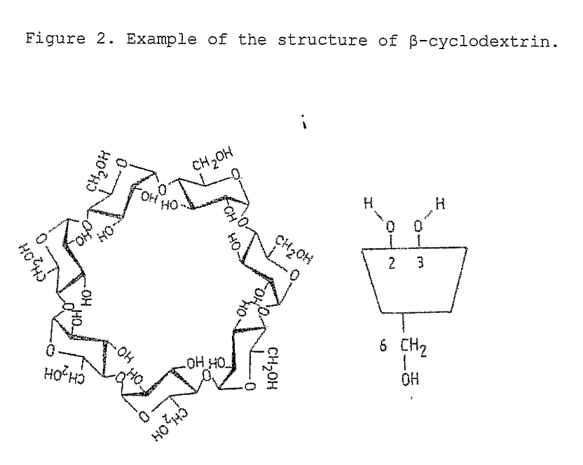

FIG. 2 represents an example of the structure of β-cyclodextrin.

In a first aspect, the present invention regards the use of a hemofilter comprising a solid support to which cyclodextrins or cyclodextrin derivatives are covalently bonded.

The solid supports employed in the invention can be polymeric resins obtained by means of a cross-linking reaction between the cyclodextrins and appropriate cross-linking agents, for example epichlorohydrin, isocyanates, polyamines, acrylates, carbonates; or fibre or fabric supports, for example cellulose, or non-woven fabric, for example polypropylene, to which the cyclodextrins are bonded by means of chemical or physical treatments such as the use of electron beam. Among these last, the preferred are the natural cellulose supports. Another support preferably employed is silica, coated with polyethylenimines derivatised with cyclodextrins.

The cyclodextrins (CD), also called cycloamyloses, cycloglucans, cyclomaltosides, are cyclic oligosaccharides constituted by the union, with α(1,4) bonds, of glucose units in a quantity which varies from 6 to 12. The word CD is preceded by a Greek letter which indicates the number of glucose units present in the ring (a corresponds to 6 units, β corresponds to 7 units, etc.).

The cyclodextrins are the product of the enzymatic degradation of the amide, by the enzyme glycosiltransferase (CGTase) produced by different bacteria (examples: Bacillus macerans, Klebsiella pneumonite, Bacillua stearothermophilus, Bacillus megaterium etc.).

The CGTase enzyme breaks the helical structure of the amide and causes the simultaneous formation of α(1,4) bonds between the glucose molecules, which lead to the obtainment of cyclic oligoglucosides.

The cyclodextrins, as shown in FIGS. 1 and 2, have a truncated cone structure with the hydroxyl groups towards the outside and the carbon, hydrogen atoms and hetero-oxide bonds towards the inside of the structure. Moreover, the primary hydroxyl groups are positioned in the zone of the smaller-diameter truncated cone and the secondary hydroxyl groups in the greater-diameter truncated cone zone.

This structure confers particular properties to the CDs: the high electron density, caused by the glycosidic oxygens, makes the cavity of the hydrophobic molecules apolar and confers a hydrophilic character to the exterior.

The principle underlying the invention is therefore that of binding the water-soluble cyclodextrins to a solid support. Blood is passed over said support, in particular blood plasma. The hydrophobic inner cavity of the cyclodextrins holds the bacterial endotoxins and thus permits the detoxification of the blood which is then reinserted clean into circulation.

The detoxification is possible in that the endotoxins or lipopolysaccharides are hydrophobic molecules and thus form a stable and reversible complex with the hydrophobic inner cavity of the cyclodextrins, called inclusion complex.

In most cases the ratio between the CD and the hosted molecule is 1:1 and there are no covalent bonds, while equilibrium is established of associative-dissociative type, due mainly to Van der Waals interactions and hydrogen bonds between the included molecule and the hydroxyls of the CD.

For the objects of the invention, the cyclodextrins preferably used are the alpha, beta, gamma cyclodextrin and their derivatives such as hydroxypropyls, sulphates and ethylsulfonates. The most preferred cyclodextrin is the alpha cyclodextrin.

The synthesis of the solid polymeric support occurs by making the cyclodextrins react with appropriate cross-linking agents. Said cross-linking agents are advantageously bifunctional molecules having a functional group at the two ends capable of reacting with the primary and secondary hydroxyls of the cyclodextrins (for example epoxides and halides).

Examples of cross-linking agents are epichlorohydrin, isocyanates, polyamines, acrylates, and carbonates; preferably epichlorohydrin is used. The synthesis method is known in literature, for example in the following article: E. Renard, G. Barnathan, A. Deratani and B. Sebille, “Characterization and structure of cyclodextrin-epichlorohydrin polymers-effects of synthesis parameters, (1996), Proceeding of the Eighth International Symposium on Cyclodextrins.

The synthesised polymers are in granular form, insoluble in water and in most organic solvents (alcohol, chloroform, acetone, DMF, DMSO etc.).

The polymers preferably used for the objects of the invention are polymers obtained from the cross-linking of cyclodextrins with epichlorohydrin, having cyclodextrin content in the range of 200-900 μmol/g, preferably 600-800 μmol/g.

The grafting of the cyclodextrins on yarn or fabric, for example cellulose, or on non-woven fabric in polymeric material, for example cellulose acetate, polypropylene, polyethylene or polyester, occurs by interposing a linker monomer between the substrate and the cyclodextrins.

There are different monomers which can be employed in the grafting reaction, for example glycidyl methacrylate (GMA), acrylic acid, N-vinylpyrrolidones, acrylamides and vinyl acetate. Preferably, the used monomer is glycidyl methacrylate.

The GMA monomer is of particular interest due to the presence of an extremely reactive group, such as the epoxide.

The method employed for the grafting of the monomer on the substrate is described in the literature, for example in the following articles: P. Le Thuaut, G. Crini, M. Morcellet, A. Naggi, U. Maschke, C. Vecchi, X. Coqueret, G. Torri and B. Martel, J. Appl. Polym. Sci., (1997); P. Le Thuaut, Macromolecular and Organic Chemistry Doctoral thesis, University of Science and Technology of Lille, Macromolecular and Chemistry Laboratory, “Fonctionnalisation de supports textiles pour l'elaboration de filtres adsorbeurs de polluants organiques”, (2000). The synthesis typically comprises the following steps:
Activation of the solid substrate by means of chemical or physical treatment, for example with electron beam etc. The preferred technique is the irradiation with electron beam;
Radical grafting reaction of the monomer which leads both to the derivatisation of the substrate and the monomer polymerisation, generating spacers of different length.

Once the spacer is grafted on the substrate, the functionalisation of the latter occurs with the cyclodextrin. The functionalisation methods with CDs of fabrics employed in the present invention are described in the literature, for example in the following articles: K. Poulakis, H. J. Buschmann and E. Schollmeyer, Patent DE 40 35378 A1, (1992); H. Reuscher and R. Hirsenkorn, EP 0 697 415 A1, (1995); H. Reuscher and R. Hirsenkorn, Patent DE 19 520 967, (1995); P. Le Thuaut, G. Crini, M. Morcellet, A. Naggi, U. Maschke, C. Vecchi, X. Coqueret, G. Torri and B. Martel, J. Appl. Polym. Sci., (1997). In a second aspect, the present invention regards a method for the detoxification of the blood comprising the following steps:
a) Drawing the blood from a patient at risk of sepsis;
b) Separating the plasma from the remaining part of the blood, inserting, on the arterial line of the extracorporeal circuit, a plasma filter of polysulfone or its derivatives, for example polyethersulfone;
c) Filtering the plasma on the hemofilter according to the invention;
d) Reuniting the filtered plasma with the previously separated blood part.

Once step d) has been concluded, the blood thus detoxified can be immediately transferred back to the patient.

In a third aspect, the invention regards the use of a hemofilter and detoxification method of the blood as described above, in the case of blood intoxication caused by the improper intake of several drug classes, for example barbiturates, or of other poisonous substances. These substance, like the lipopolysaccharides, form complexes with the supported cyclodextrins and hence are removed from the blood.

### EXPERIMENTAL DATA

### Synthesis of the Polymeric Support Material

The cyclodextrins can be polymerised by making one of the hydroxyl groups react with epichlorohydrin, bifunctional molecule. In a basic environment, the epichlorohydrin can react with the CD (cross-linking reaction) and/or with itself (homopolymerisation), as shown in the reaction diagram 1, leading to the synthesis of the polymer shown in diagram 2.

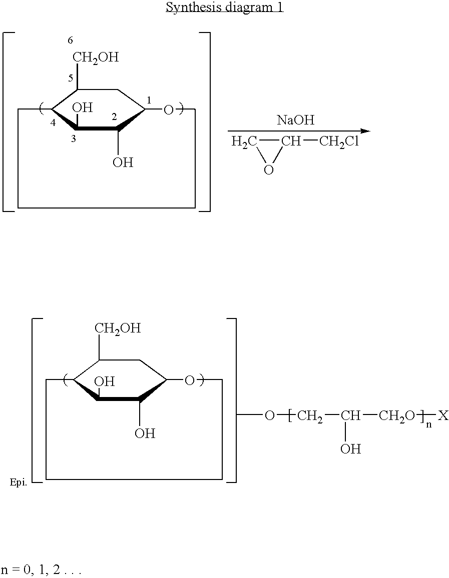

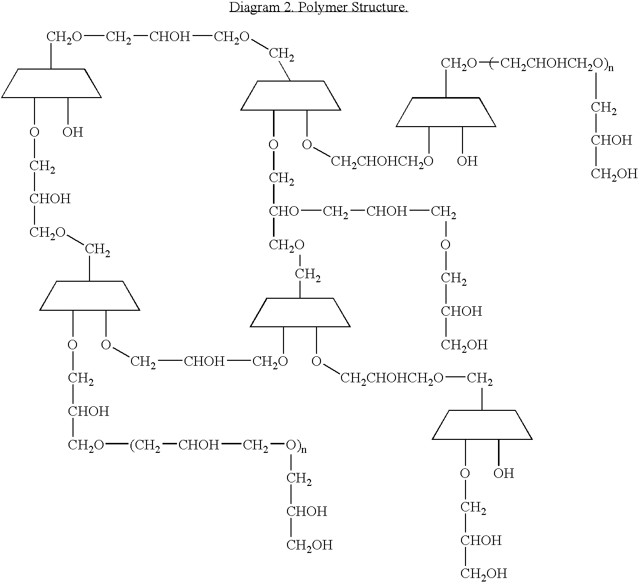

In a thermostatic reactor, a mechanical stirrer mixes different percentages of cyclodextrin (Wacker) and NaOH (Carlo Erba) in aqueous solution. After an hour of stirring, the desired amount of epichlorohydrin (Fluka) is slowly dripped, and the formation of a whitish paste of high viscosity is immediately observed, which is maintained under vigorous stirring for different times as described in table 1. Then, acetone (Acros) is added and the product is recovered for gooch filtration. The excess non-reacted epichlorohydrin and cyclodextrin is eliminated by means of washing of the polymer with hot water and ethanol (Girelli) in Soxhlet. Finally, the recovered product is dried by means of lyophilisation.

For the determination of the CD present in the synthesis products, the glucose, obtained by a total hydrolysis of the polymers, is quantified with colourimetric metering (employing phenol).

To such end, 20 mg of polymer is suspended in 5 ml of 1 M trifluoroacetic acid (Fluka), the suspension is heated at 120° C. for eight hours under vigorous stirring. After which, the solution is evaporated to eliminate the trifluoroacetic acid. The product is recovered in 10 ml of distilled water. The glucose content is determined by means of colourimetric metering with phenol and sulphuric acid. The method requires a calibration line: to such end, solutions are prepared with difference glucose concentration (Fluka) as shown in the table, beginning with a mother solution of 1% glucose by weight (1 g/l).

| Glucose solution volume (μl) | Water volume (μl) |
|---|---|
| 0 | 500 |
| 20 | 480 |
| 30 | 470 |
| 40 | 460 |

The same procedure is repeated for the hydrolysed polymer solution. In fact, different volumes are drawn from the 10 ml neutral solutions, diluted with different quantities of water as reported in the table:

| Volume glucose solution to | |
|---|---|
| be metered (μl) | Water volume (μl) |
| 20 | 480 |
| 50 | 450 |
| 100 | 400 |

To these solutions, placed in test tubes, 0.5 ml of a 5% by weight aqueous phenol solution (Carlo Erba) is added. After 10 minutes, 2.5 ml of 98% H₂SO₄ is added (Carlo Erba), vigorously stirring. After another ten minutes after this addition, the absorbance is read at the wavelength of 480 nm. Using the Lambert-Beer law, it is possible to build the calibration line and therefore determine the glucose content in the hydrolysed polymer solution. In this manner the cyclodextrin content is obtained, expressed as μmoles or μg per g of material.

The calculation of the cyclodextrin content in the synthesised polymers has permitted dividing the materials into two groups: with low (˜300 μmol/g) and high (˜600/800 μmol/g) CD content.

The different experimental conditions and the obtained CD values are reported in table 1.

TABLE 1
Description of the obtained polymers

| | | Epi./CD | | | | | | |
|---|---|---|---|---|---|---|---|---|
| | CD | (molar | NaOH Vol | | Time | T | % CD | |
| Sample | (g.) | ratio) | (23% w/w) | | (h) | (° C.) | (w/w) | μmol/g |
| Beta-20 | 3 | 20/1 | 80 | ml | 24 | 80 | 20 | 176 |
| Beta-57 | 3 | 20/1 | 4 | ml | 5 | 50 | 57 | 502 |
| Beta-60 | 3 | 20/1 | 4 | ml | 2 | 50 | 60 | 527 |
| Beta-70 | 3 | 20/1 | 3 | ml | 5 | 50 | 70 | 617 |
| Beta-80 | 3 | 60/1 | 3 | ml | 24 | 50 | 80 | 705 |
| Beta-84 | 350 | 20/1 | 500 | ml | 24 | 50 | 84 | 740 |
| Gamma-73 | 350 | 15/1 | 150 | ml | 4 | 50 | 73 | 562 |
| | | | (50% | w/w) | | | | |
| Alpha-31 | 350 | 15/1 | 150 | ml | 2 | 50 | 31 | 319 |
| | | | (50% | w/w) | | | | |

The material, indicated in the table as beta-20, was synthesised by using the method known in literature (E. Renard, G. Barnathan, A. Deratani and B. Sebille, “Characterization and structure of cyclodextrin-epichlorohydrin polymers-effects of synthesis parameters, (1996), Proceeding of the Eighth International Symposium on Cyclodextrins).

During both the characterisation and calculation studies of the complexing capacities, this material was taken as reference.

From an analysis of the experimental conditions employed for the synthesis, it is concluded that the fundamental parameter for the obtainment of high cyclodextrin content materials is the quantity of solvent present in the reaction mixture.

Synthesis of the Solid Supports in Fabric and Non-Woven Fabric.

The fabrics are prepared according to the method described in “Grafting of cyclodextrins onto polypropylene nonwoven fabrics for the manufacture of reactive filters. II Characterization” B. Martel, P. Le Thuaut, G. Crini, M. Morcellet, A. Naggi, U. Maschke, S. Bertini, C. Vecchi, X. Coqueret, and G. Torri J. Appl. Polym. Sci., 78: 2166-2173, (2000) according to the diagram described in FIG. 3. The non-woven fabric is physically activated by using 5-30 Mrad with an apparatus which provides a basic activation dose by 50 KGy (5 Mrad) electron beam; to obtain greater doses the irradiation is repeated. The grafting reaction of the glycidyl methacrylate occurs by immerging the activated fabric in an aqueous bath containing the monomer in concentration from 1-70%, and heating in inert atmosphere at 70° C. for times which vary from 20-200 minutes. The amount of bound GMA is calculated from the increase in weight of the fabric after extended washings and drying. The derivatisation with cyclodextrin occurs by immerging the functionalised fabric in a solution of cyclodextrin in DMF/H₂O and heating to 80° C. for 8-24 h. The quantity of cyclodextrin introduced in calculated from the increase in weight of the fabric after extended washings and drying.

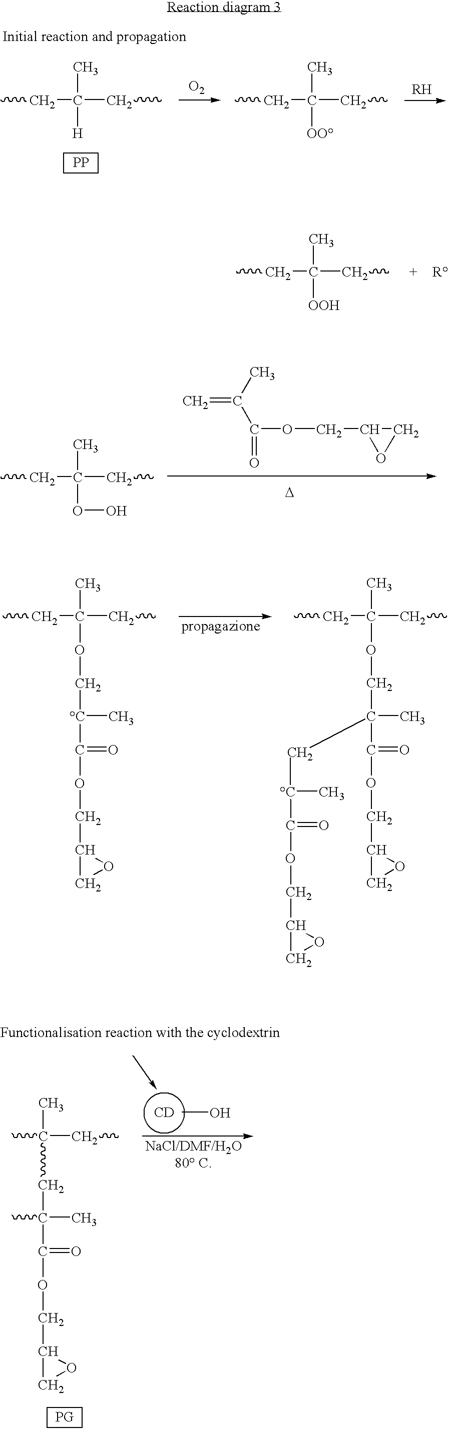

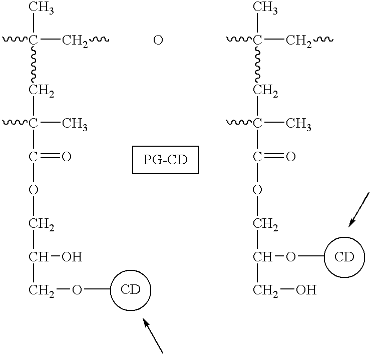

Examples of Biological Activity of the Supported Cyclodextrins of the Invention.

### Example 1

### Batch Absorption from Aqueous solutions

The following material types were tested: cyclodextrins cross-linked with epichlorohydrin (P1776: Gel beta CD; P1780: Gel gamma CD; P1793: Gel alpha CD), silica coated with polyethylenimines derivatised with CD (SiPEICD), polypropylene support derivatised with beta cyclodextrin (P1708).

100 mg of each material containing rehydrated and washed CD, is left stirring with 5 ml of a (10 mg/L) LPS solution in distilled water for 2 ore. 222 mg of P1708 are left stirring, for two hours, with 3 ml of solution.

The solutions are recovered by centrifugation at 5000 rpm for 5 minutes. From the supernatants, filtered on 1.6 μc glass fibre filters, different aliquots are drawn. The first (0.5 ml) is analysed with the phenol/sulphuric acid method, the second (5 ml) is lyophilised, and after having been taken up in 1.6 ml metered according to the Carbocyanine Dye Assay method.

Phenol/Sulphuric Acid Method

To the sample aliquots (500 μl), added to 5001 of 5% phenol in water, 2 ml of concentrated sulphuric acid is added; after 20 minutes of incubation, upon delicate stirring the absorbance is read at a wavelength of 480 nm.

Carbocyanine Dye Assay Method

### Reagents:

### 1) Carbocyanine dye

2) pH4 0.03 M Na-acetate buffer
3) 1,4-dioxane: pH4 0.03 M Na-acetate buffer (1:1 vol/vol)
4) 0.01 M Ascorbic acid

To prepare the dye solution, the reagent 1 is added to the reagent 3 in 1:2 ratio (w/vol); when solubilisation is completed, the reagent 2 is added in 1:4 (vol/vol) ratio, and finally the reagent 4 is added in 1:0.01 (vol/vol) ratio.

For the assay, the lyophilised aliquot is taken up in 0.4 ml of the reagent 2 and water is added up to a volume of 1 ml.

After stirring, 0.6 ml of dye solution is added, and it is left in incubation at room temperature for 5 minutes. The reading of the absorbance is carried out at the wavelength of 460 nm (Johnson K. G. “Isolation and purification of lipopolysaccharides” in “Methods in carbohydrate chemistry” edited by BeMiller J. N. Whistler R. L. Shaw D. H.).

As a comparison, samples of material obtained in analogous conditions are tested in the same conditions, but without the derivatisation with cyclodextrins (P1458; SiPEI;). In table 2 the retained LPS data is reported.

TABLE 2

| | CD μmol/g | % retained LPS | | |
|---|---|---|---|---|
| | (% w/w | | Carbo- | retained L [symbol] |
| | CD) | Phenol | cyanine | μg/CD μmol |
| RESINS | | | | |
| P1458 cross-linked | 0 | 0 | | 0 |
| CMC (white) | | | | |
| P1793 cross-linked | 319 (31) | 29 | | 0.114 |
| alphaCD | | | | |
| P1776: cross-linked | 740 (84) | 8 | | 0.027 |
| betaCD | | | | |
| P1780: cross-linked | 562 (73) | 52 | | 0.231 |
| gammaCD | | | | |
| SiPEI: resin (white) | 0 | | 39 | |
| SiPEI CD: betaCD | 32 (7) | | 60 | 6.450 |
| resin | | | | |
| FILTERS | | | | |
| P1708 | 176 (25) | 60 | | 0.465 |
| polypropylene/ | | | | |
| GMA/betaCD | | | | |

[symbol]

indicates data missing or illegible when filed

### Example 2

### Batch Absorption by 0.15 M NaCl Solutions

Based on the obtained results, the resins P1776, P1780, P1793 and P1708 were retested in aqueous LPS solution and in a 0.15M NaCl solution. In this case, 200 mg of P1793 was treated, compared with 100 mg of P1776 and P1780 left stirring with 5 ml of a (10 mg/L) LPS solution in distilled water for 2 hours.

Regarding P1708, 228 mg were weighed and left stirring with 3 ml of 50 μg/ml LPS in 0.15 M NaCl for 2 hours.

Also in this case, the solutions are recovered by centrifugation at 5000 rpm for 5 minutes. Two aliquots (500 μl) were metered of each with the phenol/sulphuric acid method described in example 1.

TABLE 3
(solution in 0.15M NaCl)

| | | % | |
|---|---|---|---|
| | μmol/g | retained | |
| | CD (% | LPS | Retained LPS |
| | w/w CD) | Phenol | μg/CD μmol |
| RESINS | | | |
| P1793: cross-linked | 319 | 51 | 0.235 |
| alphaCD | (31) | | |
| P1776: cross-linked | 740 | 46 | 0.189 |
| betaCD | (84) | | |
| P1780: cross-linked | 562 | 42 | 0.231 |
| gammaCD | (73) | | |
| FILTERS | | | |
| P1708 polypropylene/ | 176 | 20 | 0.074 |
| GMA/betaCD | (25) | | |

The materials based on cyclodextrin cross-linked with epichlorohydrin have shown good absorption values and it may be assumed that there was no contribution from the crosslink of the cross-linking agent, given by the lack of absorption capacity shown by the cross-linked white CMC (P1458). The following absorption capacity scale was observed: beta<gamma<alpha, CD quantities in the material being equal. An analogous result was observed in the presence of ionic force.

### Example 4

### Absorption in Low Pressure Column

200 mg of resin P1793 was left to rehydrate overnight and then washed with 300 ml of H₂O in column. 20 ml of the 10 μg/ml LPS solution was passed through the resin, and 3 ml (experiment I) and 1 ml (experiment II) portions were collected. Each portion was lyophilised and metered in the presence of Carbocyanine as described in example 1. In tables 4 and 5 the concentrations of LPS in μg/ml in each portion are respectively reported for the first and second experiment. An initial effectiveness of the column and its progressive saturation is noted.

TABLE 4

| Portion | LPS (μg/ml) |
|---|---|
| 1 | 3 |
| 2 | 2 |
| 3 | 6 |
| 4 | 7 |

TABLE 5

| Portion | LPS (μg/ml) |
|---|---|
| 0 | 2 |
| 1 | 6 |
| 2 | 2 |
| 3 | 3 |
| 4 | 0 |
| 5 | 2 |
| 6 | 2 |
| 7 | 4 |
| 8 | 3 |
| 9 | 4 |
| 10 | 6 |
| 11 | 3 |
| 12 | 5 |
| 13 | 3 |
| 14 | 4 |
| 15 | 4 |
| 16 | 2 |
| 17 | 3 |
| 18 | 4 |
| 19 | 3 |

### Example 5

### Absorption in High Pressure

790 mg of P1780 resin was packed in the HPLC column, washed and rehydrated with distilled H₂O for 5 hours. 5 ml of the 20 μg/ml LPS solution was passed through the resin in continuous recirculation for 30 minutes, and then an aliquot (500 μl; solution 2) was metered in double dosage, with the phenol/sulphuric acid method described in experiment method 1. For comparison, an aliquot was also metered of the LPS solution before the recirculation (solution 1). In table 6 the metered LPS concentrations in μg/ml are reported. The 60% absorption of the LPS is shown.

TABLE 6

| Solution | LPS (μg/ml) |
|---|---|
| 1 | 24 |
| 2 | 14.51 |

The same solutions were tested with the LAL-test method (CAMBREX). “Optimal” dilutions were prepared to take advantage of the sensitivity threshold of the method, foreseen at 0.125 EU/mL. The results are reported in table 7.

TABLE 7

| | Sol. 1 | Sol. 2 |
|---|---|---|
| | start | Column |
| Dilution | 20 mg/L LPS | gammaCD - End |
| 1:50000 | Positive | Positive LAL- |
| | LAL-test | test |
| 1:100000 | Positive | LAL-test +/− |
| | LAL-test | |
| 1:200000 | Positive | Negative LAL- |
| | LAL-test | test |
| 1:400000 | LAL-test +/− | LAL-test |
| | | negative |

Observations: the comparison between the two preparations shows a different positive result with the test for two proportional dilutions; it may therefore be hypothesised that in the sample treated with gamma-CD there was a removal of LPS equal to about 60%.

### ADVANTAGES

The use of supported cyclodextrins for the production of a hemofilter for the selective removal of the lipopolysaccharides from the blood permits considerably reducing the cost of the single hemofilter, to the great advantage of the health of persons affected by sepsis: cyclodextrins are compounds which can be easily and thus economically supplied.

Moreover, the effectiveness of detoxification of the blood is much greater than that of other currently-available filters.

The use of supported cyclodextrins permits carrying out a single filtration of the plasma in order to obtain a complete elimination of the endotoxins, unlike the detoxifying systems of the prior art which require repeated applications to reach the same result.

1. Use of cyclodextrins supported on solid support for the preparation of a hemofilter for the removal of the lipopolysaccharides in the blood.

2. Use according to claim 1 wherein said solid support is a polymeric resin.

3. Use according to claim 2 wherein said polymeric resin is cyclodextrin cross-linked with a cross-linking agent chosen from among: epichlorohydrin, isocyanates, polyamines, acrylates, carbonates.

4. Use according to claim 2 wherein said polymeric resin is cyclodextrin cross-linked with epichlorohydrin with a cyclodextrin content in the range of 200-900 μmol/g.

5. Use according to claim 4 wherein said cyclodextrin content is in the range of 600-800 μmol/g.

6. Use according to claim 1 wherein said solid support is silica covered with polyethylenimines.

7. Use according to claim 1 wherein said solid support is yarn or fabric or non-woven fabric.

8. Use according to claim 7 wherein said fabric is cellulose and said non-woven fabric is polypropylene, polyethylene, polyester, cellulose acetate.

9. Use according to claim 7 wherein said non-woven fabric is polypropylene.

10. Use according to claim 9 wherein said cyclodextrin is bound to the yarn or fabric or non-woven fabric through a linker monomer.

11. Use according to claim 10 wherein said linker monomer is chosen from among: glycidyl methacrylate (GMA), acrylic acid, N-vinylpyrrolidones, acrylamides and vinyl acetate.

12. Use according to claim 11 wherein said linker monomer is glycidyl methacrylate (GMA).

13. Use according to claim 1 wherein said cyclodextrins are chosen from among: alpha cyclodextrin, beta cyclodextrin, and gamma cyclodextrin, preferably alpha cyclodextrin.

14. Use according to claim 1, in cases of intoxication caused by the improper intake of poisonous substances and/or drugs.

15. Method for the detoxification of the blood comprising the following steps:
a. Drawing the blood from a patient at risk of sepsis;
b. Separating the plasma from the remaining part of the blood;
c. Filtering the plasma with the supported cyclodextrins according to claim 1; and
d. Reuniting the filtered plasma with the previously separated blood part.

16. Method according to claim 15 which comprises, after step d), a step of transferring the purified blood back to the patient.

17. Method according to claim 15 wherein the bacterial endotoxins and exotoxins are removed from the blood.

18. Method according to claim 17 wherein said bacterial endotoxins and exotoxins are lipopolysaccharides of gram-negative bacteria.

19. Method according to claim 15 for the prevention of the sepsis syndrome.

20. Method according to claim 15 for the removal from the blood of poisonous substances and/or drugs, such as barbiturates.
